(19) United States
(12) Patent Application Publication
(10) Pub. No.: US 2010/0178285 A1
(43) Pub. Date: Jul. 15, 2010

(54) SYNERGISTIC ANTHELMINTIC COMPOSITION

(76) Inventors: Chandrakant Laxminarayan Rathi, Maharashtra (IN); Shilpa Prasanna Risbud, Maharashtra (IN)

Correspondence Address: LADAS & PARRY LLP, 224 SOUTH MICHIGAN AVENUE, SUITE 1600, CHICAGO, IL, 60604, US

(21) Appl. No.: 12/376,695

(22) PCT Filed: Aug. 8, 2007
(86) PCT No.: PCT/IN07/00343

§ 371 Date: Feb. 6, 2009

(30) Foreign Application Priority Data
Aug. 8, 2006 (IN) 1249/MUM/2006

(51) Int. Cl. A61K 38/47; A61P 33/10

(52) U.S. Cl. 424/94.61

(57) ABSTRACT

The present invention discloses novel synergistic anthelmintic compositions used for prevention and treatment of gastrointestinal nematodes comprising combination of therapeutically effective amount of ginger extract with proteolytic enzyme and fibre degrading enzymes. The novel anthelmintic compositions provide synergistic effect at comparatively lower doses which are affordable and impart minimal/no side effect on general health as well as milk yield of dairy animals.

### TECHNICAL FIELD OF THE INVENTION

The present invention relates to novel synergistic anthelmintic compositions used for prevention and treatment of gastrointestinal nematodes comprising combination of therapeutically effective amount of ginger extract with proteolytic enzyme and fibre degrading enzymes. The novel anthelmintic compositions of the present invention when administered preferably as oral dosage form serves as non synthetic nutritional feed supplement and works as a health rejuvenator in dairy animals.

### BACKGROUND AND PRIOR ART OF THE INVENTION

Livestock are an important and integrated component of the agricultural production system in developing countries. Livestock are domesticated animals intentionally reared in an agricultural setting to produce food or fibre or for their labour. Livestock include pigs, cattle, goats, deer, sheep, yaks and poultry. They are reared under a wide variety of production systems ranging from large-scale intensive commercial farms to traditional smallholder and village production systems. Like in other developing countries, particularly in Asia, majority of the farmers in India raise their livestock under traditional production as a sideline to the main agricultural activities. However, the livestock production plays a significant role in supporting farmer's income.

As most of the developing countries of the world lie in tropical and subtropical region, warm and humid climatic conditions in the tropic/subtropics provide favourable environment for development of worm eggs to infective larvae almost throughout the year. Thus helminth parasite problem is unquestionably a major limiting factor in the improvement of livestock production.

The uses of plant and animal parts for medicines has been in existence for long time and are widely documented in records kept in ancient China, India and Egypt. These ancient indigenous practices were discovered by a series of “trial and error methods” which then could not be substantiated by proven scientific theories. However, these practices have produced results of proven efficacies compared to conventional modern medicine (Chopra et al., 1956). In recent times, herbal medicines have become indispensable and are forming an integral part of the primary health care system of many nations. United States of America (USA) (1977) indicate an expected 20% annual growth in herbal medicines with an estimated 80% of the world population living in the developing countries still relying on plants for health care.

In view of this large dependence on traditional health practices, the World Health Organization (WHO) recognized the implicit role of herbal medicine in the Alma Mata declaration of Health for All by the Year 2000 A.D. In 1978, WHO approved the use of these natural products. In Nigeria, Fulani herdsmen and others who keep animals as a means of livelihood have been involved in the treatment of animal diseases prior to the onset of modern medicine (Nwude, 1986), of which remedies against parasitism account for the highest means of intervention (Ibrahim et al., 1984).

For the above mentioned reasons, one should not neglect the fact that there is a long tradition of ethno-veterinary remedies and practice for the most common animal disease including GIT (Gastro-intestinal tract) parasite infection.

Atessahin A, Karahan I, Pirincci I. Department of Pharmacology and Toxicology, Faculty of Veterinary Medicine, Firat University, 23119 Elazig, Turkey have studied the effects of therapeutic and toxic doses of levamisole on thyroid hormones and some biochemical parameters in sheep. This study was carried out to establish the effects of therapeutic and toxic doses of levamisole on thyroid hormone levels and some biochemical parameters in sheep. Twelve Akkaraman ewes were used. Levamisole was given orally at doses of 7.5 mg kg(−1) (group 1) and 40 mg kg(−1) (group 2) to the animals. Blood samples were taken from the jugular vein at 2, 4, 8, 24, 48, 96 and 144 h after the administrations. Serum thyroid hormones and some biochemical parameters were determined on these samples. When compared with the control levels, no significant changes were observed in triiodothyronine (T3) and thyroxin (T4) levels in group 1. Although levamisole was found to increase the levels of total T3, it decreased the levels of total T4 in group 2. On the other hand, free T3 and free T4 levels were not changed in either group. While serum alkaline phosphatase (ALP) activities were decreased, aspartate aminotransferase (AST), alanine aminotransferase (ALT), lactate dehydrogenase (LDH) and creatinine kinase (CK) activities were increased significantly by levamisole. However, it increased the serum albumin and cholesterol levels, but decreased the inorganic phosphate levels in groups 1 and 2 on the other hand, when compared with the control levels. In conclusion, therapeutic and toxic doses of levamisole were determined to affect thyroid metabolism and some biochemical parameters in sheep. (Parasitology, 2004 August; 129(pt 2): 245-53)

Efficacy of albendazole and levamisole against gastrointestinal nematodes of sheep and goats in Morogoro, Tanzania has been studied by Keyyu J D, Mahingika H M, Magwisha H B, Kassuku A A. Department of Veterinary Microbiology and Parasitology, Faculty of Veterinary Medicine, Sokoine University of Agriculture, PO Box 3019, Morogoro, Tanzania. A study was conducted to determine the efficacy of albendazole after it had been withdrawn from use due to the development of resistant strains of nematodes about ten years ago. The study also aimed to determine the present efficacy of levamisole, which had been recommended to replace albendazole. On one farm, the sheep and goats were divided into two groups, one group of each serving as the untreated control, while the other was treated with levamisole. The sheep on the other farm were divided into three groups, one serving as the untreated control group, the second being treated with levamisole and the third being treated with albendazole. Faecal samples were collected one day before treatment, and again 10 days after treatment. Anthelmintic efficacy was determined by the faecal egg count reduction test. Ten days after treatment, the sheep treated with levamisole on the first farm had a 98% reduction in faecal egg count, with a 95% confidence limit of 76%. The goats on the same farm had a 97% reduction in faecal egg count, with a 95% lower confidence limit of 81%. At the second farm, 10 days after treatment, sheep treated with levamisole had a 99.4% reduction in faecal egg count, with a 95% lower confidence limit of 88.9%, whereas the sheep treated with albendazole only had a 59.4% reduction in faecal egg count, with a 95% lower confidence limit of −19.6%. The study indicated that the gastrointestinal nematodes of sheep at the Department of Animal Science and Production farm were still resistant to albendazole about ten years after this anthelmintic had been withdrawn from use. A reduced efficacy of levamisole was suspected. (Trop Anim Health Prod. 2002 March; 34(2):115-20)

In Department of Veterinary Parasitology, University of Agriculture, Faisalabad 38040, Pakistan.Iqbal Z, Lateef M, Akhtar M S, Ghayur M N, Gilani A H did the research work on in-vivo anthelmintic activity of ginger against gastrointestinal nematodes of sheep. This paper describes the anthelmintic activity of Zingiber officinale Roscoe (family Zingiberaceae) rhizome, commonly known as ginger, to justify its traditional use in veterinary medicine. Crude powder (CP) and crude aqueous extract (CAE) of dried ginger (1 kg) were administered to sheep naturally infected with mixed species of gastrointestinal nematodes. Both CP and CAE exhibited a dose- and a time-dependent anthelmintic effect with respective maximum reduction of 25.6% and 66.6% in eggs per gram (EPG) of faeces on day 10 of post-treatment.

Levamisole (7.5 mg/kg), a standard anthelmintic agent, exhibited 99.2% reduction in EPG. This study shows that ginger possesses in vivo anthelmintic activity in sheep thus justifying the age-old traditional use of this plant in helminth infestation. (J. Ethnopharmacol. 2006 Jan. 26; 106(2) 285-7)

Assessment of the anthelmintic effect of natural plant cysteine proteinases against the gastrointestinal nematode, Heligmosomoides polygyrus, in vitro has been evelulated by Stepek G, Buttle D J, Duce I R, Lowe A, Behnke J M. School of Biology, University Park, University of Nottingham Nottingham NG7, 2RD,UK. They examined the mechanism of action and compared the anthelmintic efficacy of cysteine proteinases from papaya, pineapple, fig, kiwi fruit and Egyptian milkweed in vitro using the rodent gastrointestinal nematode Heligmosomoides polygyrus. Within a 2 h incubation period, all the cysteine proteinases, with the exception of the kiwi fruit extract, caused marked damage to the cuticle of H. polygyrus adult male and female worms, reflected in the loss of surface cuticular layers. Efficacy was comparable for both sexes of worms, was dependent on the presence of cysteine and was completely inhibited by the cysteine proteinase inhibitor, E-64. LD50 values indicated that the purified proteinases were more efficacious than the proteinases in the crude latex, with purified ficin, papain, chymopapain, Egyptian milkweed latex extract and pineapple fruit extract containing fruit bromelain, having the most potent effect. The mechanism of action of these plant enzymes (i.e. an attack on the protective cuticle of the worm) suggests that resistance would be slow to develop in the field. The efficacy and mode of action make plant cysteine proteinases potential candidates for a novel class of anthelmintics urgently required for the treatment of humans and domestic livestock. (Parasitology. 2005 February; 130(Pt2): 203-11.)

Japanese patent JP1013995 discloses collection and processing of a proteolytic enzyme papain from green papaya fruit which has high hydrolyzing ability and used as digesting agent, clearer for removing clouding in beer or soy sauce, anthelmintic agent, etc.

GB 190705078 discloses a medicine for the treatment of disorders of the stomach, liver, digestive organs, or blood in cattle, sheep for destroying worms, consisting of aniseed, santonine, sulphate of iron, linseed oil, ginger, bay salt, sulphate of copper, starch, quassia chips, and water.

GB682343 discloses a proteolytic enzyme having a disulphide group in its molecule, together with cysteine or a salt thereof and a solid diluent of the type commonly used in pharmacy in the production of tablets, pills and dragees. Such compositions have anthelmintic properties. An example is given of papain with cysteine hydrochloride.

GB2413764 discloses a method for treatment of anthelmintic infections in animals and a synergistic anthelmintically effective composition consisting of at least one compound selected from each of the following groups; macrocylic lactones, benzimidazoles, salicylanilides and imidazothiazoles and a therapeutically acceptable carrier.

Control of gastrointestinal helminth infections in the livestock relies mainly on the use of anthelmintics in combination with farm management. Unfortunately in many developing countries people cannot apply anthelmintic control program using commercial modern anthelmintics for the following reasons.
Drugs are unavailable in rural areas or their supply is erratic.
Imported drugs are expensive.
Many stock raisers either under dose to save money, or over dose because they do not understand the instructions for use.
Commercial anthelmintic available in the market is usually packed for large number of animals (50-100 heads), which is more than the average number of animal property in each family.
Development of anthelmintic resistance, due to continuous long term drug application, which has also become a threat to continuing livestock production throughout the tropics/subtropics (Waller, 1998).
Serious side effects of Synthetic drugs.

Therefore, in view of the aforementioned drawbacks associated with prior art compositions of anthelmintics, it is apparent that there exists a need for compositions which are effective, economic and devoid of side effects.

### OBJECT OF THE INVENTION

The main object of the present invention is to provide novel synergistic anthelmintic compositions used for prevention and treatment of gastrointestinal nematodes comprising combination of therapeutically effective amount of ginger extract with proteolytic enzyme and fibre degrading enzymes.

Another object of the present invention is to provide the novel anthelmintic compositions in the form of oral dosage form which serves as non synthetic nutritional feed supplement and works as a health rejuvenator in dairy animals as well as human.

As per yet another object, the novel anthelmintic compositions of the present invention are safe and possess minimal/no side effects in comparison to the available allopathic drugs used for the treatment of anthelmintics.

Further object of the present invention is to provide novel anthelmintic compositions having economic significance in comparison to the commonly available compositions.

Yet another object of the present invention is to provide novel anthelmintic compositions which do not produce any resistance in gastrointestinal nematodes or may develop resistance at a comparatively slow rate.

### SUMMARY OF THE INVENTION

The present invention discloses novel synergistic anthelmintic compositions used for prevention and treatment of gastrointestinal nematodes comprising combination of therapeutically effective amount of ginger extract with proteolytic enzyme and fibre degrading enzymes. The novel anthelmintic compositions provide synergistic effect at comparatively lower doses which are affordable and impart minimal/no side effect on general health as well as milk yield of dairy animals.

### DESCRIPTION OF THE INVENTION

The invention will now be described in detail in connection with certain preferred and optional embodiments, so that various aspects thereof may be more fully understood and appreciated.

In accordance with the above objectives, the present invention provides novel synergistic anthelmintic compositions used for prevention and treatment of gastrointestinal nematodes comprising combination of therapeutically effective amount of ginger extract with proteolytic enzyme and fibre degrading enzymes.

The novel anthelmintic compositions of the present invention when administered preferably as oral dosage form serves as non synthetic nutritional feed supplement and works as a health rejuvenator in dairy animals.

Ginger extracts normally work as anthelmintic at the dose level of 3 gm/kg body weight. Papaya fruit extracts can be administered around 1 kg to sheep as anthelmintic compositions.

In a preferred embodiment, the novel anthelmintic compositions as per the present invention is a combination of plant proteases selected from the group consisting of Papain, bromelain, fucin, fungal proteases and bacterial protease alone or in combination thereof and ginger extract along with Fiber degrading enzymes selected from the group consisting of cellulases, pectinases, xylanases, beta glucanases and hemicellulases alone or combination thereof. All these enzymes used for the purpose of present invention are originated from vegetable or microbial sources. This novel anthelmintic composition provides synergistic effect at comparatively lower doses which are affordable and impart no side effect on general health as well as milk yield of dairy animals.

The ginger extract can advantageously be used in the present compositions in a concentration of 1 gm to 10 gm.

The proteolytic enzymes can be used in a concentration range of 0.5 gm to 5.0 gm. The fiber degrading enzymes are used in a concentration of 2 gm to 10 gms.

The invented compositions can advantageously be used plant proteolytic enzyme like papain and/or ficin having proteolytic activity of 20,000 TU/GM to 1,00,000 TU/GM.

In one aspect, the anthelmintic compositions comprise cellulase as fiber degrading enzyme having cellulolytic activity of 5000 CMC U/GM to 50,000 CMCU/GM.

In another aspect, the anthelmintic compositions comprises pectinase as fiber degrading enzyme having pectinolytic activity of 10,000 AJDU/GM to 60,000 ADDU/GM.

In yet another aspect, the anthelmintic compositions may comprise a combination of cellulase and pectinase in effective amounts.

The anthelmintic compositions of the present invention do not produce any resistance in gastrointestinal nematodes.

The present invention is more specifically explained by following examples. However, it should be understood that that the scope of the present invention is not limited by the examples in any manner. It will be appreciated by any person skilled in this art that the present invention includes the following examples and further can be modified and altered within the technical concept of the present invention.

### EXAMPLES

### Example A

| Ingredient | Quantity |
|---|---|
| Ginger | 1.0 gm |
| Fiber hydrolyzing enzyme (Cellulase) | 2.5 gm |
| Plant Proteolytic enzyme (Papain) | 0.500 gm |
| Citic acid | 0.2 gm |
| Lactose | QS |

### Example B

| Ingredient | Quantity |
|---|---|
| Ginger | 1.25 gm |
| Fiber hydrolyzing enzyme(Pectinase) | 2.5 gm |
| Plant Proteolytic enzyme (Papain) | 0.625 gm |
| Citric Acid | 0.2 gm |
| Lactose | QS |

### Example C

| Ingredient | Quantity |
|---|---|
| Ginger | 2.5 gm |
| Fiber hydrolyzing enzyme(cellulose) | 05 gm |
| Plant Proteolytic enzyme (Fucin) | 1.25 gm |
| Citric Acid | 0.2 gm |
| Lactose | QS |

### Example D

| Ingredient | Quantity |
|---|---|
| Ginger | 5.0 gm |
| Fiber hydrolyzing enzyme (Pectinas + Cellulase) | 10 gm |
| Plant Proteolytic enzyme (Papain) | 2.5 gm |
| Citric Acid | 0.2 gm |
| Lactose | QS |

### Example E

| Ingredient | Quantity |
|---|---|
| Ginger | 3.5 gm |
| Fiber hydrolyzing enzyme (Pactinase) | 07 gm |
| Plant Proteolytic enzyme (Fucin) | 1.75 gm |
| Citric Acid | 0.2 gm |
| Lactose | QS |

All the above compositions were found to be stable.

Experiments:

Expt No: 1

The 50 gm of composition containing the above ingredients were given to Cattle's and Goats two times a day for one day. The result obtained is provided in a tabular format in Table 1. Table 1 indicates the Mean EPG [Egg Per Gram] of Fasciola sp. and the percentage reduction in EPG.

TABLE 1
MEAN EPG 0F FASCIOLA sp. AND PERCENTAGE REDUCTION IN EPG

| | | 1st Week | 2nd Week | 3rd Week | 4th Week |
|---|---|---|---|---|---|
| Groups | 0 Days | (PT) | (PT) | (PT) | (PT) |
| Control | 13 | 18 | 20 | 20 | 24 |
| A | 10 | 0.00 (100.0) | 1.00 (95.0) | 0.00 (100.0) | 0.00 (100.0) |
| B | 11 | 1.00 (94.0) | 2.00 (90.0) | 10.00 (50.0) | 13.00 (46.0) |
| C | 11 | 1.00 (94.0) | 0.00 (100.0) | 2.00 (90.0) | 3.00 (88.0) |
| D | 16 | 6.00 (67.0) | 2.00 (90.0) | 2.00 (90.0) | 0.00 (100.0) |
| E | 18 | 7.00 (61.0) | 7.00 (65.0) | 0.00 (100.0) | 0.00 (100.0) |

Figures in parenthesis indicate % efficacy.
PT = Post Treatment.

The data on the efficacy of the drug at different dose rates and milk yield are shown in Table 2, Table 3 and Table 4.

Efficacy {% FECRT [Faecal egg count reduction test]} was calculated by the following formula.

$$\% Efficacy = 100 \times \frac{\begin{matrix}Mean E P G of untreated control animals - \\ Mean E P G of Treated animals\end{matrix}}{Mean E P G of untreated controls} .$$

Group A: Efficacy ranged with 95% on day 14 and 100% on day 7, 21 and 28 after treatment.

Group B: An efficacy of 94.0% was seen on day 7 and then efficacy decreased to 46% on day 28 post treatment.

Group C: Faecal egg count reduction test (FECRT) showed 94.4% and 100% reduction in EPG on day 7 and 14, respectively with 90% and 88% reduction on day 21 and 28 post treatment.

Group D: The efficacy increased from 67% on day 7 to 100% on day 28.

Group E: There was 61% & 65% reduction on day 7 and 14, respectively and 100% reduction in EPG on day 21and 28 after treatment.

Table 2, Table 3 and Table 4 indicates Average EPG [Egg Per Gram] and milk yield. From week 1 to week 4.

TABLE 2

| | 0 Days | | | | 1st Week (PT) | | | | |
|---|---|---|---|---|---|---|---|---|---|
| | | | | Milk | | | | | Milk |
| | | | | Yield | | | | | Yield |
| Groups | S | F | A | (kg) | S | F | | A | (kg) |
| Control | 5 | 13 | 0 | 7.3 | 10 | 18 | | 0 | 6.8 |
| A (10) | 40 | 10 | 0 | 2.7 | 30 | 0.00 | (100) | 29 | 2.7 |
| B (10) | 10 | 11 | 5 | 4.55 | 0 | 1.00 | (94.0) | 4 | 4.67 |
| C (10) | 0 | 11 | 4 | 3.7 | 0 | 1.00 | (94.0) | 2 | 5 |
| D (10) | 5 | 16 | 0 | 5.75 | 0 | 6.00 | (67) | 6 | 8.25 |
| E (10) | 0 | 18 | 3 | 1.6 | 0 | 7.00 | (61) | 10 | 1.5 |

TABLE 3

| | 2nd Week (PT) | | | | 3rd Week (PT) | | | |
|---|---|---|---|---|---|---|---|---|
| | | | | Milk | | | | Milk |
| | | | | Yield | | | | Yield |
| Groups | S | F | A | (kg) | S | F | A | (kg) |
| Control | 10 | 20 | 0 | 6.75 | 10 | 20 | 0 | 6.75 |
| A | 35 | 1.00 (95) | 12 | 2.7 | 0 | 0.00 (100) | 10 | 2.7 |
| B | 0 | 2.00 (90) | 0 | 4.72 | 0 | 10.00 (50) | 3 | 4.72 |
| C | 0 | 0.00 (100) | 1 | 5.45 | 0 | 2.00 (90) | 3 | 5.5 |
| D | 0 | 2.00 (90) | 14 | 8.35 | 0 | 2.00 (90) | 8 | 8.35 |
| E | 0 | 7.00 (65) | 19 | 1.7 | 0 | 0.00 (100) | 0 | 1.7 |

TABLE 4

4th Week (PT)

| | | | | Milk Yield |
|---|---|---|---|---|
| Groups | S | F | A | (kg) |
| Control | 10 | 24 | 0 | 6.6 |
| A | 0 | 0.00 (100) | 0 | 2.7 |
| B | 0 | 13.00 (46) | 3 | 4.72 |
| C | 0 | 3.00 (88) | 17 | 5.65 |
| D | 0 | 0.00 (100) | 3 | 8.35 |
| E | 0 | 0.00 (100) | 0 | 1.8 |

Figures in parenthesis indicate % efficacy.
PT = Post - treatment
S = Strongly
F = Fasciola,
A = Amphistome,
(10) = Numbar of Animals in group.

TABLE 5
Hematological parameters

| | 0 Days | | | | 4 Week (PT) | | | |
|---|---|---|---|---|---|---|---|---|
| | Hb | RBC | WBC | | Hb | RBC | WBC | |
| Groups | GM/100 ML | Million/mm³ | per mm³ | PCV | GM/100 ML | Million/mm³ | per mm³ | PCV |
| Control | 12 | 6 | 6000 | 26 | 12 | 6 | 6200 | 26 |
| A (10) | 11 | 7 | 5000 | 27 | 11 | 7 | 6300 | 27 |
| B (10) | 14 | 6 | 7500 | 38 | 14 | 6 | 8202 | 38 |
| C (10) | 9 | 7 | 5030 | 42 | 9 | 7 | 7300 | 42 |
| D (10) | 12 | 8 | 6500 | 26 | 12 | 8 | 6450 | 26 |
| E (10) | 10 | 6 | 6785 | 32 | 10 | 6 | 7000 | 32 |

Discussion and Conclusion:

Animals in all groups were apparently healthy and did not show any adverse health symptoms. There was slight improvement in the hematological parameters of animals after treatment which shows that the composition acts as a health rejuvenator.

The number of milching and pregnant animals in each of all the trial groups was healthy. There was positive effect on milk yield in animals of group C and D without any increase in milk in animals of any other group hence the drug was well tolerated and no side effects were observed in any animal of all the treated groups

Prior art reveals, Ginger extract (3 gm/kg) as an effective anthelmintic while the addition of proteolytic & fiber degrading enzyme to Ginger extract synergistically reduces the dose to 0.2 gm/kg.

The proteolytic and fiber hydrolyzing enzyme are large molecular weight protein molecules and so there is no problem of resistance development.

It will be evident to those skilled in the art that the invention is not limited to the details of the foregoing illustrative examples and that the present invention may be embodied in other specific forms without departing from the essential attributes thereof, and it is therefore desired that the present embodiments and examples be considered in all respects as illustrative and not restrictive, reference being made to the appended claims, rather than to the foregoing description, and all changes which come within the meaning and range of equivalency of the claims are therefore intended to be embraced therein.

1. A cost-effective, novel and synergistic non resistant anthelmintic compositions comprising combination of therapeutically effective amount of ginger extract with at least one proteolytic enzyme and at least one fibre degrading enzymes useful for prevention and treatment of gastrointestinal nematodes with minimal or no side effects.

2. The anthelmintic compositions as claimed in claim 1, wherein the ginger extract is used in a concentration of 1 gm to 10 gm.

3. The anthelmintic compositions as claimed in claim 1, wherein the proteolytic enzymes are used in a concentration of 0.5 gm to 5.0 gm.

4. The anthelmintic compositions as claimed in claim 1, wherein the fiber degrading enzymes are used in a concentration of 2 gm to 10 gms.

5. The anthelmintic compositions as claimed in claim 1, wherein the said composition is in the form of oral dosage form which serves as non synthetic nutritional feed supplement and works as a health rejuvenator in dairy animals.

6. The anthelmintic compositions as claimed in claim 1, wherein the proteolytic enzyme is plant proteases selected from the group consisting of papain, bromelain, fucin, fungal proteases, bacterial protease or in combinations thereof.

7. The anthelmintic compositions as claimed in claim 1, wherein the fibre degrading enzyme is selected from the group consisting of cellulases, pectinases, xylanases, beta glucanases, hemicellulases or in combinations thereof.

8. The novel anthelmintic compositions as claimed in claim 6 wherein said plant proteolytic enzyme is papain and/or ficin having proteolytic activity from 20,000 TU/GM to 1,00,000 TU/GM.

9. The anthelmintic compositions as claimed in claim 7 wherein the said fiber degrading enzyme is cellulase having cellulolytic activity of 5000 CMC U/GM to 50,000 CMCU/GM.

10. The anthelmintic compositions as claimed in claim 7 wherein said fiber degrading enzyme is pectinase having pectinolytic activity of 10,000 AJDU/GM to 60,000 AJDU/GM.

11. The anthelmintic compositions as claimed in claim 1, wherein said compositions do not produce any resistance in gastrointestinal nematodes.

12. The anthelmintic compositions as claimed in claim 1, wherein said compositions do not produce any side effects.

13. The anthelmintic compositions as claimed in claim 1, wherein said compositions increase the milk yield in dairy animals.

14. The anthelmintic compositions as claimed in claim 1, wherein said compositions work as a health rejuvenator in dairy animals.

15. (canceled)
